(19) United States
(12) Patent Application Publication
(10) Pub. No.: US 2020/0214315 A1
(43) Pub. Date: Jul. 9, 2020

(54) SUGAR COATING METHOD AND SUGAR COATED SOLID FORMS WITH IRREGULAR SHAPES

(71) Applicant: ROQUETTE FRERES, Lestrem (FR)

(72) Inventors: Philippe LEFEVRE, HAVERSKERQUE (FR); Gregory LE BIHAN, ANNEZIN (FR); Sebastien CROQUET, MERVILLE (FR)

(21) Appl. No.: 16/621,816

(22) PCT Filed: Jun. 18, 2018
(86) PCT No.: PCT/EP2018/066134

§ 371 Date: Feb. 20, 2020

(30) Foreign Application Priority Data
Jun. 19, 2017 (FR) 17 55557

(51) Int. Cl. A23G 3/54; A23G 3/34; A23P 20/18; A23L 29/30; A61K 9/20

(52) U.S. Cl. CPC A23G 3/54; A23G 3/0089; A61K 9/2018; A23G 3/0095; A23L 29/37; A23P 20/18

(57) ABSTRACT

The present invention relates to new sugar-coated solid forms with irregular shapes, and to a sugar coating method which is particularly useful for the preparation of same.

### CROSS-REFERENCE TO RELATED APPLICATIONS

This application is a National Stage Application of PCT/EP2018/066134 filed Jun. 18, 2018, which claims priority from French Patent Application No. 17 55557, filed on Jun. 19, 2017. The priority of said PCT and French Patent Application are claimed. Each of the prior mentioned applications is hereby incorporated by reference herein in its entirety.

The subject of the present invention is novel sugar-coated solid forms having irregular shapes, and also a sugar-coating process which is particularly useful for the preparation of same.

### CONTEXT OF THE INVENTION

Sugar-coating is an operation used in particular in confectionary or pharmacy, which consists in creating a crystallized coating that is more or less hard (“hard” or “soft coating”) on the surface of solid or pulverulent products, so as to protect them for various reasons or to make them attractive visually or in terms of taste.

The coating of the solid form (core) is carried out in a tank rotating about its axis and known as a coating drum, inside which there is a plurality of cores forming a mass in motion, on the surface of which is distributed, in the liquid state, the constituent material of the future envelope (“sugar-coating liquid or syrup”). The hard and crystalline coating is obtained by applying this liquid and evaporating off the water introduced by said liquid.

The sugar-coating syrup is mainly composed of one or even more crystallizable materials, and also conventionally contains binding agents such as gum Arabic or gelatin, dyes, opacifiers such as TiO₂, mineral fillers such as talc, silica, calcium carbonate, intense sweeteners, flavors, vitamins, active agents.

Sugar-coating is a relatively laborious process, which includes a large number of successive steps. Each of these steps, also called “sugar-coating cycles”, typically includes an application phase, generally by spraying a sugar-coating syrup onto the cores, a rotary phase of distribution of said syrup on the cores, also called pause time, and a phase of drying each new layer of syrup carried out by blowing hot, dry air.

Sugar-coating makes it possible to obtain solid forms having a particularly attractive appearance, but lacks flexibility as to its implementation and as to the visuals that can be obtained. In particular, and this is the subject of the present invention, it is not possible in sugar-coating to obtain solid forms having complex irregular shapes, for example relief impressions or ridges. This is because, when solid forms having such irregularities are sugar coated, the latter are masked after sugar-coating: the sugar-coating liquid fills the grooves and cavities formed by the relief impressions (“logo in-filling”), and enlarges the ridges which then round off. It is also not possible to preserve the asymmetry that the cores optionally exhibit, such as for example when these cores are grains. The solid forms obtained by sugar coating thus conventionally have a coarse and rounded general shape, not very faithful to that of the starting cores.

Therefore, for example in pharmacy, film coating of tablets is generally preferred. In this process, a film-forming composition, generally based on cellulose derivatives such as hydroxypropylmethylcellulose (HPMC), or more recently, modified starches, is applied, and not a syrup. This film-forming composition forms a thin film at the surface of the tablets and advantageously makes it possible to preserve the relief impressions present at the surface of the tablets, such as logos, names of active molecules or dosages of acting molecules.

Although this process makes it possible to preserve precise shapes, it nevertheless leads to tablets which have a much less attractive appearance than the sugar-coated forms. Furthermore, the film coating produces an amorphous coating, which is much less stable than the crystallized coating resulting from the sugar coating.

Thus, at the current time, there is no process for producing sugar-coated solid forms having original shapes. Thus, when the manufacturer wishes to display logos or other patterns on the surface of the sugar-coated solid forms, it does so by means of the application of a colored composition on the surface of the sugar-coated forms.

One approach to this problem is described in document US 2008/0026131 A1. This document discloses a sugar-coating process which makes it possible to preserve the irregular shape of chocolates. However, this process only makes it possible to preserve coarse and round general shapes, and not complex irregular shapes. The teaching of document US 2008/0026131 A1 does not make it possible to prepare sugar-coated solid forms having complex irregular shapes such as ridges or relief impressions. This process, which is particularly long and laborious, comprises multiple sugar-coating cycles (applying the syrup/distribution/drying).

### OBJECTIVES OF THE INVENTION

The aim of the present invention is thus to provide sugar-coated solid forms having complex irregular shapes, and also a process which makes it possible to achieve this.

The invention also aims to provide a particularly simple and flexible process for the sugar coating of solid forms.

### PRESENTATION OF THE INVENTION

The applicant has been able to produce sugar-coated solid forms in which the irregular shapes such as relief impressions or ridges, and the asymmetrical shapes are preserved after sugar coating.

The applicant has achieved this through the implementation at least of one new sugar-coating process, characterized in that it comprises:
a step (a) of spraying a sugar-coating liquid comprising a composition of crystallizable materials onto a bed of cores in motion, said cores being placed in a chamber equipped with a perforated rotating drum, and said spraying being carried out by means of at least one compressed air nozzle;
at least one step (b), concomitant with step (a), of drying said sprayed sugar-coating liquid;
a step (c) of collecting the sugar-coated solid forms thus obtained.

The process of the invention applies to varied solid forms (tablets, chewing gums, etc.) and offers a multitude of possibilities for manufacturers, and in particular that of now being able to offer the consumer new visuals of solid sugar-coated forms.

In this process, the drying and the spraying are carried out concomitantly. In point of fact, surprisingly, despite the continuous wetting of the cores, the latter do not erode, and the irregular shapes are preserved.

This is particularly surprising since it was up until now accepted that the spraying necessarily had to be stopped for a not insignificant period of time during the process: otherwise, the cores are perpetually wetted, so that the substance used for the sugar coating cannot crystallize, that the solid form erodes and degrades, and that the cores adhere to each other (phenomenon of sticking together in lumps). However, this phenomenon is not observed with the process of the invention, even on very hygroscopic cores, that is to say water-greedy cores.

Thus, and contrary to conventional sugar coating, the process of the invention does not require the sugar-coating liquid to have a high solids content (at least 70% for conventional sugar coating with maltitol). This also goes against the general knowledge in the art for the same reasons as those mentioned in the previous paragraph (significant wetting of the cores).

Being able to use low solids contents has a certain number of advantages. Firstly, it gives the possibility of using sugar-coating liquids at low temperatures, and thus of introducing thermolabile ingredients into these liquids or into the compositions of cores to be sugar coated. Indeed, if high temperatures are conventionally used in sugar coating, it is to allow the sugar-coating solution to have a high content of crystallizable materials in solution. Since this high solids content is not necessary in the process of the invention, the temperature of the sugar-coating liquid can be lowered.

Secondly, this also makes it possible to carry out sugar-coating operations with crystallizable materials having a low solubility in water. A good illustration of this advantage is presented in the Examples under point 1. (Tests “Man-IN1 to -IN5”), wherein mannitol was used for the sugar coating. This sugar coating does not require the addition thereto of large amounts of binders, contrary to the prior art processes. In other words, a sugar coating, strictly speaking, is performed here, since the crystallizable materials can be largely in the majority.

Thus, for the first time, the process of the invention has made it possible to obtain sugar-coated solid forms of which the sugar-coating layer has high contents of mannitol, without this requiring the addition of excessive amounts of binders such as gum Arabic or polyvinyl alcohol. The test Man-IN5 even shows that it is possible to dispense with binders.

The process developed by the applicant also has other advantages which make it also useful for the sugar coating of solid forms which do not particularly have complex irregular shapes. In particular, the process of the invention is much more flexible than conventional sugar-coating processes, and can be particularly simple to implement.

In addition, this process is applicable to various crystallizable materials, as demonstrated in point 1. below in the examples. Sucrose, xylitol, erythritol and mannitol in particular give very satisfactory results.

Finally, the process of the invention does not necessarily require large modifications of its parameters during its execution (drying temperature, formulation of the sugar-coating liquid, etc.), and can be implemented in a relatively simple and compact piece of equipment.

None of the prior art documents discloses or suggests such a process. For example, the abovementioned document US 2008/0026131 A1 never simultaneously carries out drying and spraying.

### SUMMARY OF THE INVENTION

A first subject of the present invention is a sugar-coated solid form having at least one relief impression and/or at least at least one ridge, and/or in that it is asymmetrical, the sugar-coating layer of said asymmetrical sugar-coated solid form faithfully matching the shape of the core from which it is derived.

A subject of the present invention is also a process which is particularly useful for the preparation of sugar-coated solid forms having complex irregular shapes, comprising:
a step (a) of spraying a sugar-coating liquid comprising a composition of crystallizable materials onto a bed of cores in motion, said cores being placed in a chamber equipped with a perforated rotating drum, and said spraying being carried out by means of at least one compressed air nozzle;
at least one step (b), concomitant with step (a), of drying said sprayed sugar-coating liquid;
a step (c) of collecting the sugar-coated solid forms thus obtained.

A subject of the present invention is also a sugar-coated solid form comprising at least one sugar-coating layer comprising:
crystallizable materials consisting of at least 50% by dry weight of mannitol; and
optionally binders, the crystallizable materials/binders ratio by dry weight being greater than 15.

### DETAILED DESCRIPTION OF THE INVENTION

The first subject of the present invention is thus sugar-coated solid forms having at least one relief impression and/or at least one ridge, and/or in that it is asymmetrical, the sugar-coating layer of said asymmetrical sugar-coated solid form faithfully matching the shape of the core from which it is derived.

The solid forms of the invention have particular irregular shapes, that is to say relief impressions, ridges, or asymmetries. As examples of relief impressions, mention may be made of patterns (such as logos) or inscriptions (brand names, molecule names, dosages, etc.). The relief impressions are present on the surface of the solid forms, whereas the ridges instead contribute to defining the general shape of the sugar-coated solid form. These sugar-coated solid forms are in particular characterized in that the cores to be sugar-coated, from which they are derived, themselves have regular shapes. It is in particular meant that these irregular shapes of the initial cores are preserved after sugar coating. This means in particular that the inscriptions are readable, and that the patterns are identifiable. These irregular shapes also cover asymmetrical shapes, that is to say those which have no axis of symmetry. It is meant here that the sugar-coating layer of the asymmetrical sugar-coated solid forms faithfully matches the shape of the cores from which they are derived.

Preferably, this solid form of the invention has at least one relief impression and/or at least one ridge, more preferably at least one relief impression.

A subject of the present invention is also a sugar-coated solid form having the irregular shapes optionally as described above, characterized in that it comprises at least one sugar-coating layer comprising:
crystallizable materials consisting of at least 50% by dry weight of mannitol; and
optionally binders, the crystallizable materials/binders ratio by dry weight being greater than 15.

Preferably, the crystallizable materials of the sugar-coating layer of this sugar-coated solid form consists of at least 60% by dry weight of mannitol, preferably of at least 70%, preferably of at least 80%, preferably of at least 90%, or even solely of mannitol.

More preferably, in the sugar-coating layer of these sugar-coated solid forms, the crystallizable materials/binders ratio by dry weight is greater than 20, or even greater than 30, or even greater than 40. The sugar-coating layer may also advantageously be free of binders.

In sugar coating, the term “solid form” conventionally means any solid presentation form of sugar-coated substances (“sugar-coated solid form”) or solid presentation form capable of undergoing a sugar-coating operation (“core”). Typical examples are tablets, hard gel capsules, soft capsules, pellets, microspheres, granules, seeds, cookies, breakfast cereals, confectionaries such as chewing gums, boiled candies, chew pastes, jelly candies, chocolates, fruits and vegetables, or else products in the form of powders and/or of crystals. These solid forms may for example have a food, pharmaceutical, veterinary or cosmetic purpose. They may be intended for human consumption, by adults or children, or for animals. They may also be products for chemical or agrochemical purposes, although solid forms intended to be ingested are preferred in the context of the present invention. Preferably, these solid forms are chosen from tablets and chewing gums.

Preferably, the sugar-coated solid forms of the invention comprise at least one sugar-coating layer, the composition of which is identical to that of the sugar-coating liquid used in the sugar-coating process of the invention; it being understood that only what constitutes the solids of the sugar-coating liquid is being taken into consideration here.

Preferably, the sugar-coated solid forms of the invention have a sugar-coating percentage of greater than 1%. This sugar-coating percentage, otherwise known as “gain in mass”, is conventionally determined in the following way:

(Weight of sugar-coated solid forms−Weight of cores)/Weight of cores×100.

Preferably, this sugar-coating percentage is greater than 3%, preferably greater than 5%, preferably greater than 7%, preferably greater than 10%, for example at least 15%, and even at least 20%.

A subject of the invention is also a sugar-coating process which is particularly useful for the preparation of sugar-coated solid forms according to the invention, comprising:
a step (a) of spraying a sugar-coating liquid comprising a composition of crystallizable materials onto a bed of cores in motion, said cores being placed in a chamber equipped with a perforated rotating drum, and said spraying being carried out by means of at least one compressed air nozzle;
at least one step (b), concomitant with step (a), of drying said sprayed sugar-coating liquid;
a step (c) of collecting the sugar-coated solid forms thus obtained.

The equipment used to carry out the process of the invention typically comprises a unit for storing the sugar-coating liquid, comprising at least one outlet for transporting the sugar-coating liquid to a device for spraying the sugar-coating liquid. The sugar-coating liquid is applied by means of the device for spraying onto a bed of cores contained in the chamber, said chamber being equipped with a rotating drum for making said bed of cores move. More specifically, the drum is a perforated rotating drum, and the spraying device chosen comprises at least one compressed air nozzle. The equipment also comprises an air inlet at the level of the chamber of the drum, for the drying of the sugar-coating liquid. The drying air exits in particular by means of the perforations of the rotating drum, in particular by suctioning of the air from the chamber.

The elements of the equipment which is useful for the invention are commercially available, and the arrangement thereof does not represent particular difficulties for those skilled in the art.

The sugar-coating liquid which is useful for the invention comprises a composition of crystallizable materials.

The term “composition of crystallizable materials” conventionally means a composition consisting of substances capable of crystallizing by evaporation of the solvent in which they are dissolved.

Preferably, the composition of crystallizable materials of the sugar-coating liquid which is useful for the invention comprises at least one substance chosen from sugars and polyols, preferably chosen from monomers and dimers.

Advantageously, the sugars and polyols represent at least 50%, preferably at least 70%, and even more preferably at least 90% by dry weight of the composition of crystallizable materials of the sugar-coating liquid. Most preferentially, the composition of crystallizable materials of the sugar-coating liquid consists entirely of substances chosen from sugars and polyols.

In the present invention, it is understood that, when reference is made to a dry mass of compounds, this mass includes the possible impurities.

Preferably, these sugars and polyols are chosen from xylitol, sucrose, erythritol, mannitol, dextrose, isomalt, maltitol or optionally a combination thereof. Most preferentially, these sugars and polyols are chosen from sucrose, mannitol, xylitol, erythritol, or optionally a combination thereof, more preferably from sucrose, mannitol, xylitol, or optionally a combination thereof.

Preferably, the composition of crystallizable materials of the sugar-coating liquid consists entirely of a single substance. Thus, for example and advantageously, the composition of crystallizable materials which is useful for the invention is xylitol or sucrose or erythritol or mannitol or dextrose or isomalt or maltitol.

The concentration of crystallizable materials of the sugar-coating liquid is chosen in such a way that the crystallizable materials are solubilized in said sugar-coating liquid.

Typically, in particular when the sugar-coating liquid is an aqueous liquid, the sugar-coating liquid of the invention has a concentration of crystallizable materials of less than 90%, this percentage corresponding to the dry weight of crystallizable materials of the sugar-coating liquid relative to the total weight of said sugar-coating liquid. Preferably, this concentration is less than or equal to 70%, preferably less than or equal to 60%, preferably less than 60%, or even less than or equal to 55%, or even less than or equal to 50%, or even less than or equal to 45%, or even less than or equal to 40%, or even less than or equal to 35%, or even less than or equal to 30%, or even less than or equal to 25%, or even equal to 20%. This concentration is generally greater than 10%, or even at least equal to 15%, or even at least equal to 20%.

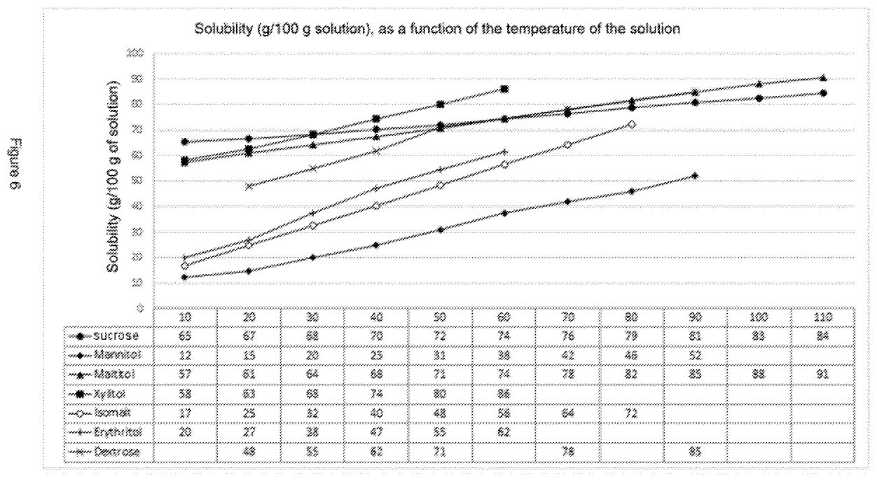

The maximum concentration of crystallizable materials naturally depends on the nature of these crystallizable materials, and also on the temperature of the sugar-coating liquid. For xylitol, sucrose, erythritol, mannitol, dextrose, isomalt and maltitol, reference may for example be made to FIG. 6 which presents the maximum concentrations of these crystallizable materials in water as a function of the temperature of the liquid. Thus, for example, the concentration of crystallizable materials of a sugar-coating liquid at 20° C. is preferentially less than or equal to 67% when the composition of crystallizable materials consists of sucrose, less than or equal to 15% when it is a question of mannitol, less than or equal to 61% when it is a question of maltitol, less than or equal to 63% when it is a question of xylitol, less than or equal to 25% when it is a question of isomalt, less than or equal to 27% when it is a question of erythritol, less than or equal to 48% when it is a question of dextrose.

Generally, the sugar-coating liquid of the invention comprises substances other than crystallizable materials, as long as that does not conflict with the desired properties in the present invention, in particular with regard to the quality of the sugar-coated solid forms obtained and/or the handlability of the process. Such other compounds are for example:
binders, such as gum Arabic, polyvinyl alcohol;
coloring substances such as pigments, opacifiers, for instance titanium dioxide;
flavorings, sweeteners;
active agents, for example of pharmaceutical, nutritive, nutraceutical or phytosanitary interest.

Preferably, the sugar-coating liquid of the invention has a solids content by weight of less than 90%, preferably less than 85%, preferably less than 80%, preferably less than 75%, preferably less than or equal to 70%, preferably less than or equal to 60%, preferably less than 60%, or even less than or equal to 55%, or even less than or equal to 50%, or even less than or equal to 45%, or even less than or equal to 40%, or even less than or equal to 35%, or even less than or equal to 30%, or even less than or equal to 25%, or even equal to 20%. This solids content is generally greater than 10%, preferably at least equal to 15%, more preferably at least equal to 20%.

Preferably, the solids content of the sugar-coating liquid of the invention thus consists of:
50.0% to 100.0% by dry weight of crystallizable materials, in particular as defined above;
from 0.0% to 50.0% by dry weight of other ingredients, in particular as defined above; the sum of these percentages being equal to 100.0%.

Preferably, the solids content of the sugar-coating liquid of the invention comprises an amount of at least 60.0% by dry weight of crystallizable materials, preferably of at least 70.0%, preferably of at least 80.0%, preferably of at least 90.0%, or even of at least 94.0%, or even of at least 95.0%, or even of at least 96.0%.

Generally, the solids content of the sugar-coating liquid of the invention comprises an amount of less than 5.0% by dry weight of pigments and/or dyes, preferably less than 4.0%, preferably less than 3.0%.

Generally, the solids content of the sugar-coating liquid of the invention comprises an amount of less than 5.0% by dry weight of opacifiers, preferably less than 4.0%, preferably less than 3.0%, preferably less than 3.0%, preferably less than 2.0%.

Generally, the solids content of the sugar-coating liquid of the invention comprises an amount of less than 15.0% by dry weight of binder, preferably less than 10.0%, or even less than 8.0%, or even less than 5.0%.

The sugar-coating liquid used in accordance with the invention is typically polar, and preferentially comprises water as major solvent, most preferentially as sole solvent.

In one advantageous embodiment, for reasons of simplicity and since it is possible, the sugar-coating process according to the invention uses a single sugar-coating liquid, that is to say that the sugar-coating liquid has a constant formulation throughout the duration of the sugar coating.

The temperature of the sugar-coating liquid is typically chosen such that the crystallizable materials are well solubilized in said sugar-coating liquid to be sprayed. This temperature thus also depends on the amount of crystallizable materials present in the liquid. In the process of the invention, this temperature is typically chosen from a range extending from 20 to 90° C.

Preferably, this temperature is less than 85° C., preferably less than 80° C., preferably less than 75° C., preferably less than 70° C., or even less than 65° C., or even less than 60° C., or even less than 55° C., or even less than 50° C., or even less than 45° C., or even less than 40° C., or even less than 35° C., or even less than 30° C. This temperature is generally at least 15° C., or even at least 20° C. It corresponds for example to ambient temperature, which typically ranges from 20 to 25° C.

In one advantageous embodiment, the sugar-coating liquid is stored in an enveloped simple storage unit, and/or the equipment which is useful for the process of the invention is free of a device for heating the sugar-coating liquid. This is because, since the process of the invention does not necessarily require the use of high sugar-coating-liquid temperatures, these devices are not obligatory in the process of the invention.

For the spraying of the sugar-coating liquid, the number of compressed air nozzles used is conventionally chosen as a function of the dimensions of the sugar-coating chamber, according to the constructor's recommendations. This number of nozzles is typically from 1 to 2 nozzle(s) per section that is 40 cm in diameter of the sugar-coating chamber. This number of nozzles ranges, for example, from 1 to 10, for example from 1 to 6.

Preferably, for the spraying, the process of the invention uses only compressed air nozzles.

Preferably, the nozzles used in accordance with the invention have an orifice which has a diameter chosen from a range extending from 0.1 to 2.8 mm, preferably from 0.1 to 2.5 mm, preferably from 0.1 to 2.2 mm, for example from 0.3 to 2.0 mm, or from 0.5 to 1.8 mm or from 0.5 to 1.5 mm, or from 0.5 to 1.2 mm, or from 0.5 to 1.0 mm.

Preferably, the spray flow rates are chosen from a range extending from 0.5 to 20.0, preferably from 1.0 to 20.0, preferably from 2.0 to 20.0, for example from 2.0 to 15.0, or even from 2.0 to 10.0 g/min/kg of cores.

In one advantageous embodiment, the flow rate chosen for the spraying is increased during the sugar coating. In particular, the inventors have noted that, for certain crystallizable materials, the use of low flow rates at the beginning of sugar coating makes it possible to promote the first crystallization phase which takes place at the surface of the cores. The flow rate can then be increased so as to accelerate the sugar coating.

For the spraying, the atomization and compression pressure is adjusted as a function of the flow rate and of the orifice of the nozzle, and according to the constructor's recommendations.

These atomization and compression pressures are typically chosen in a range extending from 0.5 to 4.0 bar, preferably extending from 0.5 to 3.5 bar, for example from 0.7 to 2.5 bar for the atomization pressure, and/or from 0.7 to 3.5 bar for the compression pressure.

In the present invention, the sugar-coating liquid is sprayed onto a bed of cores made to move by means of a rotating drum.

The nature of these cores is preferably as defined above for the solid forms; they are for example tablets or chewing gums. These cores may be completely bare, or may be coated with one or more layers, for example gumming layers, film-coating layers, or even sugar-coating layers, said layers preferentially being obtained in the same equipment as that used for the sugar-coating process of the invention.

For making the bed of cores move, the rotational speed of the drum is chosen as a function of the dimensions of the chamber and of the size of the cores to be sugar-coated. It is generally chosen in a range extending from 3 to 30 rpm, for example in a range extending from 10 to 20 rpm.

In one advantageous embodiment, in particular for reasons of simplicity, and/or of amount of space taken up, and because the process of the invention allows it, the process of making the bed of cores move excludes the transporting of said cores in a longitudinal axis. This means in particular that the cores undergoing sugar coating are not transported from one chamber to another, that is to say that they are sugar coated in a single chamber, and/or that the bed of cores is not sugar coated in a longitudinal chamber along which they are transported.

For the drying, the temperature chosen for the drying air is preferably less than 100° C., preferably less than or equal to 80° C., preferably less than or equal to 75° C., preferably less than or equal to 70° C., or even less than or equal to 65° C., or even less than or equal to 60° C., or even less than or equal to 55° C., or even less than or equal to 50° C., or even less than or equal to 45° C., or even less than or equal to 40° C., or even less than or equal to 35° C., or even less than or equal to 30° C., or even less than or equal to 25° C. This drying temperature is generally at least 15° C., preferably at least 20° C.

For the drying, the flow rate can be chosen in a range extending from 50 to 8000 m³/h, for example in a range extending from 100 to 7000 m³/h, for example from 100 to 1000 m³/h.

The drying air advantageously exits by suctioning of air by means of the perforations of the perforated rotating drum.

Preferably, the perforated wall zone of the rotating drum which is useful for the invention preferentially represents at least 50% of the surface area of said drum wall, preferably at least 60%, preferably at least 70%, preferably at least 80%, preferably at least 90%. Most preferentially, the drum wall is perforated over its entire surface area.

Preferably, the temperature of the bed of cores undergoing sugar coating is at most 70° C., preferably at most 60° C., preferably at most 55° C., preferably at most 50° C., preferably at most 45° C., or even at most 40° C., or even at most 35° C., or even at most 30° C., or even at most 25° C. This temperature of the bed of cores undergoing sugar coating is generally at least 10° C., or even at least 15° C.

Preferably, before the start of the sugar coating, that is to say before the spraying begins, the process of the invention comprises a step of heating the bed of cores to be sugar coated. This step aims in particular to bring the bed of cores to a target temperature.

The sugar-coating process of the invention concomitantly carries out spraying and drying of the spraying liquid.

However, it is possible to envision introducing steps of distribution (“pause time”) and spraying in the absence of drying, as long as this does not conflict with the desired properties in the present invention, in particular with regard to the quality of the sugar-coated solid forms obtained and/or the handlability of the process.

Preferably, the sugar-coating process phases during which the spraying is optionally not carried out concomitantly with the drying represent less than 50% of the sugar-coating process time, preferably less than 40%, preferably less than 30%, preferably less than 20%, preferably less than 10%, preferably less than 5%. Most preferentially, the sugar coating does not comprise a spraying phase in the absence of drying.

Preferably, the optional pause times (times between two spraying phases during which there is neither a spraying nor drying) represent less than 50% of the sugar-coating process time, preferably less than 40%, preferably less than 30%, preferably less than 20%, preferably less than 10%, preferably less than 5%. Most preferentially, the sugar coating does not comprise a pause time.

Preferably, the sugar-coated solid forms obtained by means of the process of the invention have a sugar-coating percentage of greater than 1%, preferably greater than 3%, preferably greater than 5%, preferably greater than 7%, preferably greater than 10%, for example of at least 15%, or even of at least 20%.

Preferentially, this sugar-coating percentage is, per unit of time, at least de 0.05% per minute, preferably at least 0.10% per minute, preferably at least 0.13% per minute, or even at least 0.15% per minute, or even at least 0.20% per minute, or even at least 0.30% per minute.

The process of the invention may also comprise usual steps other than those aimed at sugar coating the solid forms, as long as this does not conflict with the desired properties in the present invention, in particular with regard to the quality of the sugar-coated solid forms obtained and/or the handlability of the process. As an example of other steps, mention may for example be made of gumming, smoothing, polishing, coloring. Advantageously, if such steps are carried out, they are performed in the same equipment as that used in the sugar-coating process of the invention. It is understood that some of these steps, such as the gumming or the coloring for example, can sometimes be assimilated into sugar-coating steps, if the composition used comprises an amount of such crystallizable materials such that there is effectively formation of a layer of crystallized substances, in particular of sugars and/or of polyols.

With regard to these other usual steps, it is interesting to note that the sugar coating carried out according to the process of the invention already makes it possible to obtain a good level of smoothing of the solid forms. The coloring can moreover be carried out directly by means of the addition of coloring agents to the sugar-coating liquid.

The process of the invention can also comprise an initiating step. This initiating step consists in applying, one or more times, a pulverulent composition to the bed of cores to be sugar coated, or undergoing sugar coating, preferentially undergoing sugar coating. This pulverulent composition typically comprises crystalline materials. These crystalline materials are typically of the same nature as the crystallizable materials of the sugar-coating liquid, but may be different. The amount of pulverulent composition applied is typically less than 10% by weight relative to the weight of tablets, preferably less than 5%.

Preferably, when dextrose, isomalt, maltitol or a combination thereof are used, the process comprises an additional initiating step. Preferably, when sucrose, xylitol, mannitol, erythritol or a combination thereof are used, the process excludes this initiating step.

The equipment which is useful for the invention may comprise other elements conventionally used in sugar coating, provided that this does not conflict with the desired properties in the present invention, in particular with regard to the quality of the sugar-coated solid forms obtained and/or the handlability of the process.

In this regard, it is interesting to note that the prior art sometimes mentions the use of lump breakers, the objective of which is to prevent the appearance of lumps during the sugar coating. Such lump breakers are not required in the process of the invention. Thus, preferably and because it is advantageously possible in the context of the process of the invention, the chamber of the drum used in accordance with the invention is free of lump breakers.

The invention will be understood more clearly by means of the examples which follow, which are illustrative and nonlimiting.

### Examples

1. Sugar-Coating Tests in Accordance with the Invention

1.1. Equipment Used

A piece of equipment having the following characteristics was used:
perforated rotating drum: diameter 30.48 cm, volume 2 L;
anti-slip bars ×6;
1 compressed air atomization nozzle (Schlick 970/7-1 S75); orifice diameter 0.8 mm;
peristaltic pump (Watson Marlow model 323); 3-roller head (313 DM;
Tube: internal diameter 2 mm: external diameter 6 mm.

1.2. Sugar-Coating Liquids

The sugar-coating liquids tested had the following formulations (% on a dry basis):

| Ref. | GA | PVA | PVA2 | BF | N |
|---|---|---|---|---|---|
| Crystallizable materials | 96.5% | 96.5% | 94.5% | 98.5% | 95.5% |
| tested | | | | | |
| Gum Arabic* | 2.0% | — | — | — | 2.0% |
| PVA (polyvinyl alcohol) | — | 2.0% | 4.0% | — | — |
| Titanium dioxide | 1.0% | 1.0% | 1.0% | 1.0% | — |
| Blue pigment | 0.5% | 0.5% | 0.5% | 0.5% | — |
| (Blue n. 1 Lake) | | | | | |
| Natural blue pigment | — | — | — | — | 2.5% |
| (GNT) | | | | | |

*introduced in the form of a solution at 40% by weight of solids

All these sugar-coating liquids were contained in a single-shell storage tank, at ambient temperature (20-25° C.).

1.3. Cores

The following batches of cores were used as sugar-coating substrate:

Ref.

| | |
|---|---|
| Comp-SORB | 900 g of (hygroscopic) sorbitol tablets of 330 mg |
| | having relief impressions |
| Comp-MANN | 900 g of (nonhygroscopic) mannitol tablets of 330 mg |
| CG | Gum (chewing gum) comprising gum base, sorbitol, |
| | mint flavoring, etc |

1.4. Processes and Results

The conditions common to all the tests were the following: operating pressure 4 bar;

atomization and compression pressure 1.0 or 1.3 bar (chosen as a function of the viscosity of the solution, as recommended by the constructor); differential pressure −0.25 mbar; rotational speed of the drum 18 rpm; drying air flow rate 100 m³/h.

Before the start of the spraying, the bed of tablets was heated for 2 minutes in order to reach the desired temperature for the bed of cores. The spraying and the drying were then carried out concomitantly throughout the process (no pause time). At the end of sugar coating, the spraying was stopped, and the solid forms were dried for a further 2 minutes.

The following tables present the results obtained, and also the process characteristics specific to each test.

| | | | | |
|---|---|---|---|---|
| Test | Test | Test | Test | Test |
| Xyl-IN1 | Xyl-IN2 | Xyl-IN3 | Xyl-IN4 | Xyl-IN5 |

Sugar-coating liquid

| | | | | | |
|---|---|---|---|---|---|
| Crystallizable | Xylitol | Xylitol | Xylitol | Xylitol | Xylitol |
| materials | | | | | |
| Ref. | GA | PVA | BF | N | N |
| formulation | | | | | |
| % solids | 60% | 60% | 60% | 60% | 60% |

Sugar-coating process

| | | | | | |
|---|---|---|---|---|---|
| Ref. cores | Comp-SORB | Comp-SORB | Comp-SORB | Comp-SORB | CG |
| Input air | 60° C. | 60° C. | 60° C. | 60° C. | 60° C. |
| temperature | | | | | |
| (drying) | | | | | |
| Spraying flow | 3.5 → 7.5 | 3.5 → 7.5 | 3.5 → 7.5 | 3.5 → 7.5 | 3.5 → 7.5 |
| rates | g/min | g/min | g/min | g/min | g/min |
| Observation of | no sticking | no sticking | no sticking | no sticking | no sticking |
| sticking | together in | together in | together in | together in | together in |
| together in | lumps | lumps | lumps | lumps | lumps |
| lumps during | | | | | |
| the process | | | | | |
| Measured | 40° C. | 40° C. | 40° C. | 40° C. | 40° C. |
| temperature of | | | | | |
| bed of cores | | | | | |
| Process | 60 minutes | 60 minutes | 60 minutes | 60 minutes | 60 minutes |
| duration | | | | | |

Sugar-coated solid forms

| | | | | | |
|---|---|---|---|---|---|
| Gain in mass | 20.0% | 20.0% | 20.0% | 20.0% | 20.0% |
| (gain in mass/ | (0.33%/ | (0.33%/ | (0.33%/ | (0.33%/ | (0.33%/ |
| minute) | minute) | minute) | minute) | minute) | minute) |
| FIGS. | 1. a) | 1. b) | 1. c) | 1. d) | 1. e) |
| Surface feel | smooth | smooth | smooth | smooth | smooth |
| Color | homo- | homo- | homo- | homo- | homo- |
| | geneous | geneous | geneous | geneous | geneous |
| Irregular | general | general | general | general | general |
| shapes | shape very | shape very | shape very | shape very | shape very |
| | clear, relief | clear, relief | clear, relief | clear, relief | clear, relief |
| | impressions | impressions | impressions | impressions | impressions |
| | clearly | clearly | clearly | clearly | clearly |
| | identifiable | identifiable | identifiable | identifiable | identifiable |

| | | | | |
|---|---|---|---|---|
| Test | Test | Test | Test | Test |
| Man-IN1 | Man-IN2 | Man-IN3 | Man-IN4 | Man-IN5 |

Sugar-coating liquid

| | | | | | |
|---|---|---|---|---|---|
| Crystallizable | Mannitol | Mannitol | Mannitol | Mannitol | Mannitol |
| materials | | | | | |
| Ref. | GA | GA | PVA | PVA2 | BF |
| formulation | | | | | |
| % solids | 20% | 20% | 20% | 20% | 20% |

Sugar-coating process

| | | | | | |
|---|---|---|---|---|---|
| Ref. cores | Comp-MANN* | Comp-SORB | Comp-SORB | Comp-SORB | Comp-SORB |
| Input air | 25° C. | 55° C. | 55° C. | 55° C. | 55° C. |
| temperature | | | | | |
| (drying) | | | | | |
| Spraying flow | 2.2 → 8.5 | 3.5 → 7.5 | 3.5 → 7.5 | 3.5 → 7.5 | 3.5 → 7.5 |
| rates | g/min | g/min | g/min | g/min | g/min |
| Observation | no sticking | no sticking | no sticking | no sticking | no sticking |
| of sticking | together in | together in | together in | together in | together in |
| together in | lumps | lumps | lumps | lumps | lumps |
| lumps during | | | | | |
| the process | | | | | |
| Measured | 21-26° C. | 35° C. | 35° C. | 35° C. | 35° C. |
| temperature | | | | | |
| of bed of | | | | | |
| cores | | | | | |
| Process | 144 minutes | 150 minutes | 150 minutes | 150 minutes | 150 minutes |
| duration | | | | | |

Sugar-coated solid forms

| | | | | | |
|---|---|---|---|---|---|
| Gain in mass | 18.9% | 20.0% | 20.0% | 20.0% | 20.0% |
| (gain in mass/ | (0.13%/ | (0.13%/ | (0.13%/ | (0.13%/ | (0.13%/ |
| minute) | minute) | minute) | minute) | minute) | minute) |
| FIGS. | 2.a) | 2.b) | 2.c) | 2.d) | 2.e) |
| Surface feel | smooth | smooth | smooth | smooth | smooth |
| Color | homo- | homo- | homo- | homo- | homo- |
| | geneous | geneous | geneous | geneous | geneous |
| Irregular | General | general | general | general | general |
| shapes | shape very | shape very | shape very | shape very | shape very |
| | clear | clear, relief | clear, relief | clear, relief | clear, relief |
| | | impressions | impressions | impressions | impressions |
| | | clearly | clearly | clearly | clearly |
| | | identifiable | identifiable | identifiable | identifiable |

*these cores did not comprise relief impressions

| | |
|---|---|
| Test Sac-IN1 | Test Ery-IN1 |

Sugar-coating liquid

| | | |
|---|---|---|
| Crystallizable materials | Sucrose | Erythritol |
| Ref. formulation | GA | GA |
| % solids | 50% | 25% |

Sugar-coating process

| | | |
|---|---|---|
| Ref. cores | Comp-SORB | Comp-SORB |
| Input air temperature | 70° C. | 70° C. |
| (drying) | | |
| Spraying flow rates | 3.5 → 6 g/min | 3.5 → 7.5 g/min |
| Observation of sticking | slight sticking together | no sticking together |
| together in lumps during | at the beginning, | in lumps |
| the process | which decreases as | |
| | the process proceeds | |
| Measured temperature of | 44-45° C. | 44-45° C. |
| bed of cores | | |
| Process duration | 70 minutes | 120 minutes |

Sugar-coated solid forms

| | | |
|---|---|---|
| Gain in mass | 20.0% | 20.0% |
| (gain in mass/minute) | (0.29%/minute) | (0.17%/minute) |
| FIGS. | 3.a) | 4.a) |
| Surface feel | +/−smooth | smooth |
| Color | homogeneous | homogeneous |
| Irregular shapes | general shape very | general shape very |
| | clear, relief | clear, relief |
| | impressions clearly | impressions clearly |
| | identifiable | identifiable |

| | |
|---|---|
| Test Iso-IN1 | Test Mal-IN1 |

Sugar-coating liquid

| | | |
|---|---|---|
| Crystallizable materials | Isomalt | Maltitol |
| Ref. formulation | GA | GA |
| % solids | 25% | 33% |

Sugar-coating process

| | | |
|---|---|---|
| Ref. cores | Comp-MANN* | Comp-MANN* |
| Initiating | 40 g of isomalt after | 50 g of maltitol after |
| | 20 g of sprayed sugar- | 35 g of sprayed sugar- |
| | coating liquid then | coating liquid |
| | 30 g of isomalt at | |
| | 40 g of sprayed | |
| | solution | |
| Input air temperature | 60° C. | 55° C. |
| (drying) | | |
| Spraying flow rates | 1.8 → 10.0 g/min | 1.7 → 13.3 g/min |
| Observation of sticking | no sticking together | sticking together |
| together in lumps during | in lumps | in lumps at the |
| the process | | beginning, which |
| | | decreases as the |
| | | process proceeds |
| Measured temperature of | 35-40° C. | 30-36° C. |
| bed of cores | | |
| Process duration | 138 minutes | 76 minutes |

Sugar-coated solid forms

| | | |
|---|---|---|
| Gain in mass | 19.5% | 15.0% |
| (gain in mass/minute) | (0.14%/minute) | (0.20%/minute) |
| Surface feel | rough | rough |
| Color | homogeneous | homogeneous |
| Irregular shapes | general shape very | general shape very |
| | clear | clear |

*these cores did not comprise relief impressions

1.5. Discussion

The process of the invention advantageously makes it possible to sugar coat all types of solid forms according to a very simple process, and makes it possible to successfully preserve complex irregular shapes, such as relief impressions, ridges, and asymmetrical shapes.

The inventors have thus succeeded for example in sugar coating tablets, and chewing gums, whether they are hygroscopic (“Comp-SORB” and “CG”) or nonhygroscopic (“Comp-MANN”) while at the same time preserving the relief impressions present at their surface.

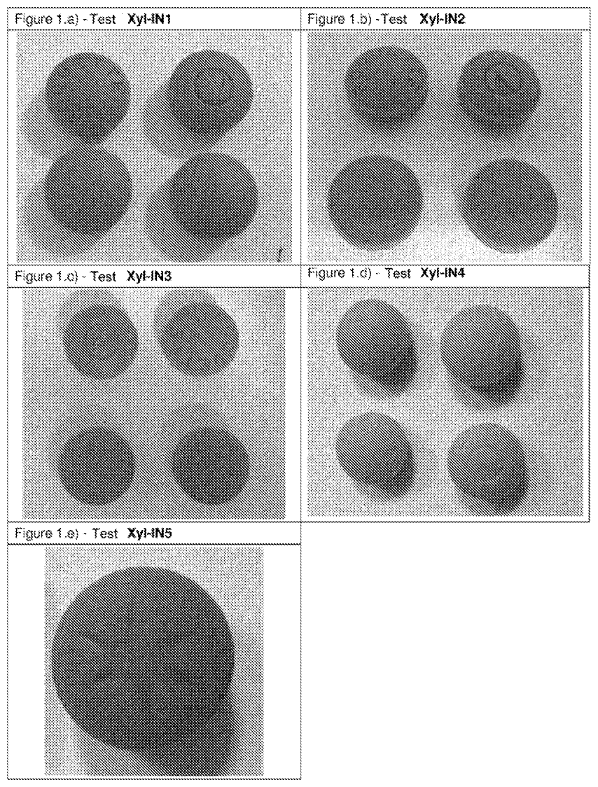

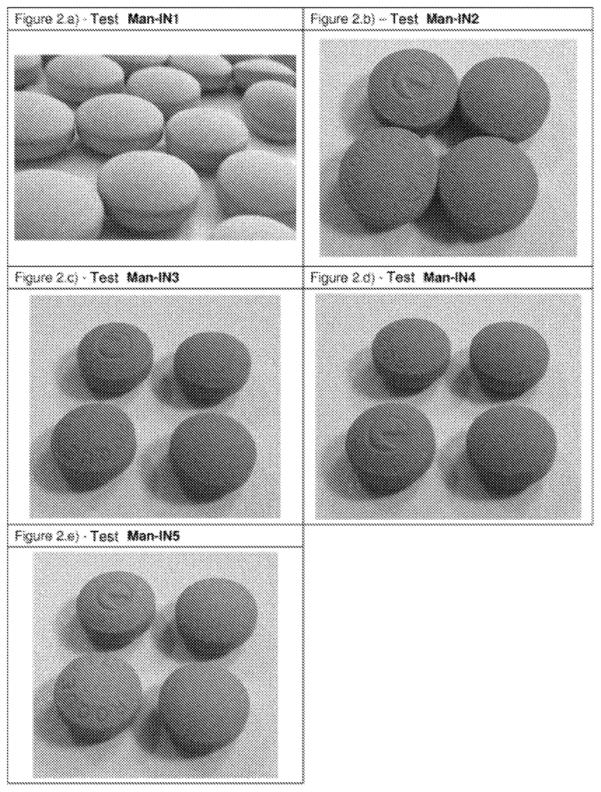

Surprisingly, despite the continuous wetting of the cores, the latter did not erode and the complex irregular shapes were preserved (absence of “logo in-filling”, that is to say filling of the grooves and cavities formed by the relief impressions). For example, in the Tests “Man-IN1 to -IN5”, a sugar-coating liquid containing 20% solids only was used. In point of fact, the sugar-coated tablets obtained have clearly visible irregular shapes (FIG. 2a), in particular relief impressions that are perfectly identifiable (FIGS. 2.b) to 2.e)).

The process of the invention also has other, not insignificant, advantages which make it likewise useful for sugar coating solid forms having conventional shapes.

The sugar-coating liquids used can contain all types of crystallizable materials and can be very simple in terms of formulation. The Tests “Xyl-IN3” and “Man-IN5” for example show that a binder-free (“BF”) sugar-coating liquid is also suitable for the process of the invention. The process of the invention also makes it possible to successfully sugar coat solid forms with “natural” sugar-coating liquids, which in particular are free of titanium dioxide (Test “Xyl-IN4”, using the sugar-coating liquid “N”).

The process of the invention makes it possible, moreover, to use very low solids contents, which is also unheard of in sugar coating. All the tests presented here use sugar-coating liquids of which the solids content does not exceed 60%. The process of the invention thus makes possible the use of carbohydrates with low solubility in water (for example mannitol) for the sugar coating.

The process of the invention also makes it possible to use relatively low drying temperatures. The main objective of the Test “Man-IN1” is to illustrate this effect and said test shows that sugar coating with mannitol using a temperature of 25° C. is possible. The sugar-coated solid forms obtained have a very clear shape, faithful to that of the starting cores (FIG. 2.a). The possible use of particularly low drying temperatures makes it possible in particular to obtain a core-bed temperature which is also low, thus making possible the sugar coating of compositions of cores having thermolabile ingredients, as is the case with certain active agents, in particular pharmaceutical active agents. Thus, in the test “Man-IN1”, the temperature of the bed of tablets does not exceed 26° C.

The temperature of the sugar-coating liquid can also be very low, since the process of the invention does not necessarily require the latter to contain a large amount of crystallizable materials in solution. In these tests, the sugar-coating liquids used were in fact at ambient temperature (20-25° C.). In addition to facilitating the implementation of the process, this makes it possible to use thermolabile ingredients in these liquids.

Finally, the solid forms obtained by means of the process of the invention are also satisfactory with regard to their color and their surface feel.

2. Comparative Sugar-Coating Tests

In this part, the inventors attempted to sugar coat 1 kg of Comp-SORB tablets having relief impressions according to the abovementioned document US 2008/0026131 A1.

2.1. Equipment used

A piece of equipment having the following characteristics was used:
rotating drum: diameter 30 cm;
anti-slip bars ×8;
1 compressed air atomization nozzle (BINKS 460); orifice diameter 0.8 mm;
peristaltic pump (Watson Marlow model 323); 3-roller head (313 DW);
tube: internal diameter 2 mm: external diameter 6 mm.

2.2. Sugar-Coating Liquids

In accordance with document US 2008/0026131 A1, example 1, the sugar-coating liquids used were the following:
“gum syrup”: formulation (% on a dry basis): 96.0% sucrose; 4.0% gum Arabic (introduced in the form of a solution at 40% by weight of solids); solids content: 72%.
“smoothing syrup”: formulation (% on a dry basis): 97.0% sucrose; 2.0% glucose syrup; 1.0% titanium dioxide; solids content: 70%.
“color syrup”: formulation (% by dry weight): 98.0% sucrose; 2.0% blue pigment (Blue n. 1 Lake); solids content: 69%.

The temperature of these liquids was adjusted if necessary so as to completely solubilize the compounds.

2.3. Processes and Results

In the tests which follow, the conditions of example 1 of document US 2008/0026131 A1 were adhered to (drying temperatures, solids, etc.). The parameters such as the spraying flow rate or the rotational speed of the drum were adjusted to the size of the sugar-coating chamber available to the inventors.

Test Sac-CP1:

A first test was carried out in accordance with the following table by performing several cycles each consisting of a spraying phase, then a pause time (distribution), then a drying step:

| | | Spraying | | Drying | Rotational |
|---|---|---|---|---|---|
| | | (flow rate, | Pause | (° C., | speed of |
| Phases | Cycles | duration) | (duration) | duration) | the drum |
| Gumming | 1-9 | 20 g/min, | 1 min | 27° C., | 19 rpm |
| (gum syrup) | | 1 min | | 7 min | |

This test could not be completed and was stopped, because of excessive sticking together in lumps, despite a drying capacity of the equipment higher than that described in document US 2008/0026131 A1 (50 m³/h compared with 15.3 m³/h).

A 2^{nd} test was thus carried out in order to avoid this phenomenon of sticking together in lumps and to be able to reproduce example 1 of document US 2008/0026131 A1 in its entirety.

Test Sac-CP2:

Following the failure of the test Sac-CP1, the first cycle of the first phase was modified: the operator intervened in order to delump the cores, and the operating conditions were modified. This test was carried out in accordance with the following table:

| | | Spraying | | Drying | Rotational |
|---|---|---|---|---|---|
| | | (flow rate, | Pause | (° C., | speed of |
| Phases | Cycles | duration) | (duration) | duration) | the drum |
| Gumming | 1 | 20 g/min, | 1 min | 27° C., | 19 −> |
| (gum syrup) | | 1 min | | 13 min | 28 rpm |
| | 2-9 | 20 g/min, | 1 min | 27° C., | 19 rpm |
| | | 1 min | | 7 min | |
| Smoothing | 1-4 | 20 g/min, | 1 min | 30° C., | 19 rpm |
| (smoothing | | 1 min | | 6 min | |
| syrup) | 5-9 | 20 g/min, | 1 min | 30° C., | 23 rpm |
| | | 1 min | | 6 min | |
| Coloring | 1-8 | 40 g/min, | 1 min | 25° C., | 28 rpm |
| (color syrup) | | 22.5 s | | 6 min | |
| | 1-3 | 40 g/min, | 4 min | 25° C., | 28 rpm |
| | | 9 s | | 30 s | |

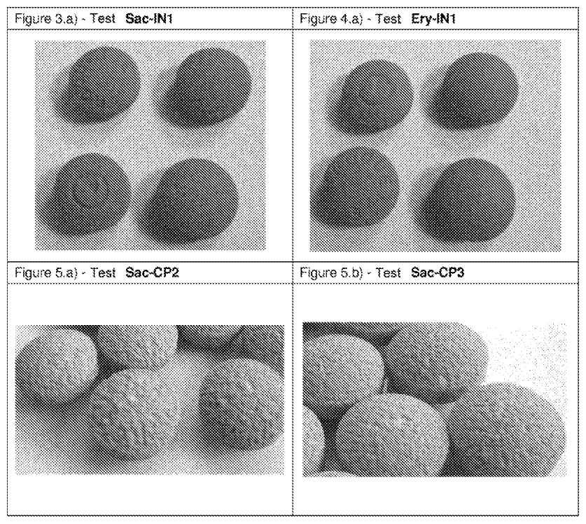

The results obtained are presented in FIG. 5.a). The tablets thus sugar coated are very rough, and have a coarse and rounded general shape. The logos and impressions that were present on the cores are covered and are no longer identifiable. The inventors noted that the surface of the tablet became damaged from the first cycles of sugar coating. The tablets solubilized from the first two minutes, despite the high solids content of the sugar-coating liquids used and the crystallization was, moreover, very difficult.

Test Sac-CP3:

A third test was carried out, in which only the “Smoothing” sugar-coating phase of the Test Sac-CP2 was carried out. Results similar to those of the Test Sac-CP2 were obtained (FIG. 5.b)): the sugar-coated tablets had a coarse and rounded general shape, and the relief impressions were covered.

1. A sugar-coated solid form, characterized in that it has at least one relief impression and/or at least one ridge and/or in that it is asymmetrical, the sugar-coating layer of said asymmetrical sugar-coated solid form faithfully matching the shape of the core from which it derives.

2. The sugar-coated solid form as claimed in claim 1, characterized in that it is chosen from tablets, hard gel capsules, soft capsules, pellets, microspheres, granules, seeds, cookies, breakfast cereals, confectionaries, and products in the form of powders and/or of crystals.

3. A sugar-coating process, comprising:
a step (a) of spraying a sugar-coating liquid comprising a composition of crystallizable materials onto a bed of cores in motion, said cores being placed in a chamber equipped with a perforated rotating drum, and said spraying being carried out by means of at least one compressed air nozzle;
at least one step (b), concomitant with step (a), of drying said sprayed sugar-coating liquid;
a step (c) of collecting the sugar-coated solid forms thus obtained.

4. The sugar-coating process as claimed in claim 3, characterized in that said composition of crystallizable materials comprises at least one substance chosen from sugars and polyols.

5. The sugar-coating process as claimed in claim 4, characterized in that the sugars and polyols represent at least 50%, preferably at least 70%, and even more preferentially at least 90% by dry weight of the composition of crystallizable materials of the sugar-coating liquid.

6. The sugar-coating process as claimed in claim 4, characterized in that said sugars and polyols are chosen from xylitol, sucrose, erythritol, mannitol, dextrose, isomalt, maltitol or a combination thereof.

7. The sugar-coating process as claimed in claim 6, characterized in that said sugars and polyols are chosen from xylitol, sucrose, erythritol, mannitol, or a combination thereof.

8. The sugar-coating process as claimed in claim 3, characterized in that said sugar-coating liquid has a concentration of crystallizable materials of less than 90%, this percentage corresponding to the weight of crystallizable materials of the sugar-coating liquid relative to the total weight of said sugar-coating liquid.

9. The sugar-coating process as claimed in claim 3, characterized in that the compressed air nozzles have an orifice having a diameter chosen from a range extending from 0.1 to 2.8 mm.

10. A sugar-coated solid form, characterized in that it comprises at least one sugar-coating layer comprising:
crystallizable materials consisting of at least 50% by dry weight of mannitol; and
optionally binders, the crystallizable materials/binders ratio by dry weight being greater than 15.
